(19) United States
(12) Patent Application Publication
(10) Pub. No.: US 2013/0116798 A1
(43) Pub. Date: May 9, 2013

(54) BONE ADHESIVE AND A METHOD OF DELIVERY

(71) Applicants: David Franklin Farrar, York (GB); David Nmi Langton, York (GB)

(72) Inventors: David Franklin Farrar, York (GB); David Nmi Langton, York (GB)

(73) Assignee: SMITH & NEPHEW PLC, Memphis, TN (US)

(21) Appl. No.: 13/808,645

(22) PCT Filed: Jul. 8, 2011
(86) PCT No.: PCT/GB11/01032

§ 371 Date: Jan. 7, 2013

(30) Foreign Application Priority Data
Jul. 9, 2010 (GB) 1011552.5
Jul. 8, 2011 (GB) PCT/GB2011/001032

(51) Int. Cl. A61F 2/28

(52) U.S. Cl. CPC A61F 2/28
USPC 623/23.62

(57) ABSTRACT

An adhesive precursor and method of delivering an adhesive precursor for fixing bone fragments together.

The present invention concerns the repair of damaged tissue, particularly bone fractures. In particular, the present invention relates to bone adhesives and their methods of delivery.

Previous attempts to develop bone adhesives have included the use of epoxy resins, polyurethanes, cyanoacrylates, polymethylmethacrylate, fibrin, gelatine-resorcinol-formaldehyde, polypeptides, calcium phosphates and glass ionomer cements.

Glass-ionomer cement (GIC) systems typically involve mixing two or more powders with an aqueous component to form an adhesive paste. In a typical preparation, the two GIC powders are pre-mixed and, when required, the mixture is activated by stirring with the aqueous component. The mixing may either be done by hand, or, more typically for glass-ionomer cements, the two components are packaged within separate chambers of a capsule. Usually, the two components are separated by a breakable foil seal. When the GIC is required, the foil is broken, typically by means of a plunger, which allows the powder and liquid to mix. The mixture is then agitated, usually in a suitable mixing device, and the mixed cement subsequently injected out of the capsule using the plunger.

Glass-ionomer cements tend to have very short setting times once they have been activated. This provides a limited working time, making them difficult materials for a surgeon to work with. Once activated, a surgeon will tend to want an adhesive to set quickly—typically within 1-2 minutes. Having said this, adhesives which have such short setting times also have very short working times, which makes them very difficult for the surgeon to handle. Conversely, an adhesive that has a good working time will generally have a long setting time, which is also undesirable.

The present invention seeks to overcome at least some of the above issues by providing an adhesive precursor with enhanced handling and mechanical properties.

In its broadest sense the invention provides an adhesive precursor and a method of delivering an adhesive precursor for fixing bone fragments together.

In a first aspect, the present invention provides an adhesive precursor for securing bone to bone, wherein the adhesive precursor is directly applicable to the site of action and is activated in-situ by endogenous aqueous fluids.

Suitably, the adhesive precursor is a glass ionomer cement, calcium phosphate, zinc polycarboxylate, polyurethane or other water-activated adhesive ceramic or polymer. Preferably, the adhesive precursor is a glass ionomer cement.

Suitably, the adhesive precursor is a powder and may be formed, with or without bulking agents, as a tablet or a pressed sheet. Alternatively, the adhesive precursor is a viscous liquid.

The adhesive precursor requires no mixing, which allows the user to utilise very reactive adhesive systems and materials with short setting times that would otherwise be impractical. It is particularly advantageous for systems that otherwise require mixing of a powder and liquid such as the glass-ionomer cement systems. The invention avoids the need for the surgeon to mix any components, and helps to simplify and reduce the amount of time required for the surgical procedure.

In a second aspect, the present invention provides a substrate comprising an adhesive precursor for securing bone to bone, wherein the substrate and adhesive precursor are directly applicable to the site of action and the adhesive precursor is activated in-situ by endogenous aqueous fluids.

Suitably, the substrate is a tape, film, paper, foil or bandage. Suitably, the substrate is a fabric. Suitably, the fabric is knitted, woven, spun or non-woven, for example felt or electrospun mat. The extent of any undesired spread of adhesive precursor around the site of application can easily be limited and controlled by cutting an appropriately sized piece of the tape or bandage, or by using an appropriately sized tablet.

Suitably, the substrate may be bioabsorbable or non-resorbable. Suitable absorbable substrates include polylactides, polyglycolides, poly(lactide-co-glycolides), polycaprolactones, polyurethanes, or other bioresorbable polymer, and ceramics or glasses such as Bioglass, calcium phosphate, or other bioabsorbable ceramics/glasses. Suitable non-resorbable substrates include polyethylene terephthalates, polyethylenes, polyurethanes, polytetrafluroethylenes, or other non-resorbable biocompatible polymers, and ceramics or glasses such as Bioglass, calcium phosphate, or other non-bioresorbable ceramics/glasses.

The adhesive precursor may be applied to one side or both sides of the substrate as a continuous coating or as a non-continuous coating. Additionally, the substrate and/or adhesive precursor may include holes to encourage new bone tissue growth between the bone fragments. By using an appropriate substrate with holes then the problem of totally occluding the healing bone ends can be avoided—such holes or gaps helping to facilitate the growth of new bone tissue. Similarly, applying the substrate on the side of the bone also helps to avoid this problem.

Alternatively, the substrate is a removable carrier formed of a film, foil or paper. The provision of a removable carrier is advantageous as it is a convenient means with which to handle and deliver the adhesive precursor to the site of application. In use, the carrier can be used to apply the adhesive precursor, as a pressed sheet or film of powder or viscous liquid, allowing pressure to be applied so that the adhesive precursor is correctly located. Once activation of the adhesive precursor has begun and the adhesive starts to form, the carrier can be peeled away and disposed of.

According to the invention, the adhesive precursor is activated by an endogenous aqueous environment around the site of application. Optionally, it may be desirable to also apply additional aqueous solution, for example by dipping a substrate including an adhesive precursor in an aqueous solution, in instances where there is a very limited amount of endogenous fluid present at the site of application so that the adhesive can properly form. Suitably, the additional aqueous solution is a saline solution, water or an aqueous solution containing an activator. Suitably, the activator is tartaric acid.

Optionally, the adhesive precursor further comprises a porogen to encourage tissue growth into/through the adhesive. Suitable examples of porogens include water soluble salts, sugars, polymers, such as, for example, sodium chloride, sucrose and polyethylene glycol.

Optionally, the adhesive precursor and/or substrate further comprises a bioactive component that stimulates tissue healing and, in particular, bone growth. Preferred examples of the bioactive component include antimicrobial compounds, growth factors such as bone morphogenetic proteins, enzymes, proteins or small molecules such as bisphosphonates.

The present invention also provides a method of combining bone fragments wherein the method comprises the steps of:
a) providing an adhesive precursor according to either the first or second aspects above; and
b) (i) applying the adhesive precursor directly to one or more of the bone fragments to be combined, and
   (ii) bringing the bone fragments together; or
c) (i) bringing the bone fragments together, and
   (ii) applying the adhesive precursor to one or more outer surfaces of the bone fragments to be combined;
      wherein the adhesive precursor is activated in-situ by endogenous aqueous fluids which are present at the site of application.

Optionally, where the adhesive precursor is formed with or on a substrate, the adhesive precursor is dipped in an aqueous solution prior to its application to the bone fragments. This may be advantageous in sites of application where a very limited amount of endogenous fluid is present.

The above and other aspects of the invention will now be described with reference to the following drawings in which:

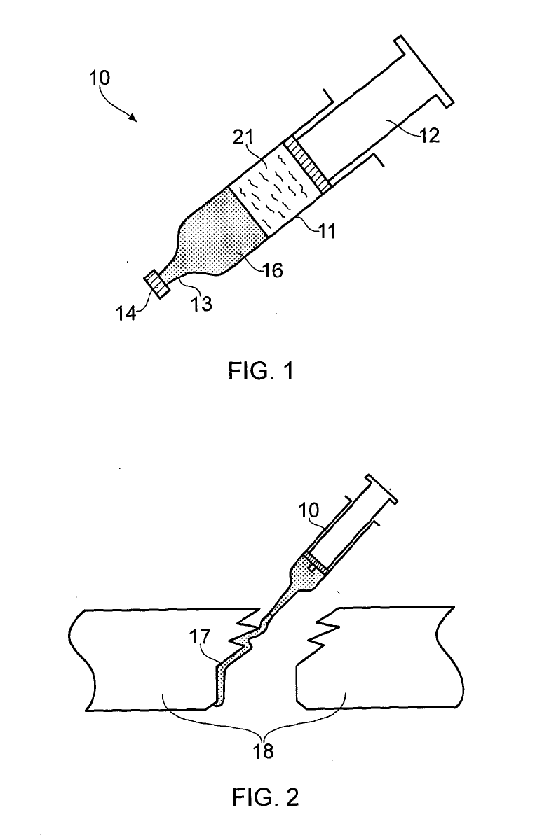

FIG. 1 is a schematic adhesive delivery device of the prior art;

FIG. 2 is the prior art delivery device of FIG. 1 shown in-situ;

FIGS. 3A&B are schematic side views of an embodiment of a substrate including an adhesive precursor according to the second aspect of the invention;

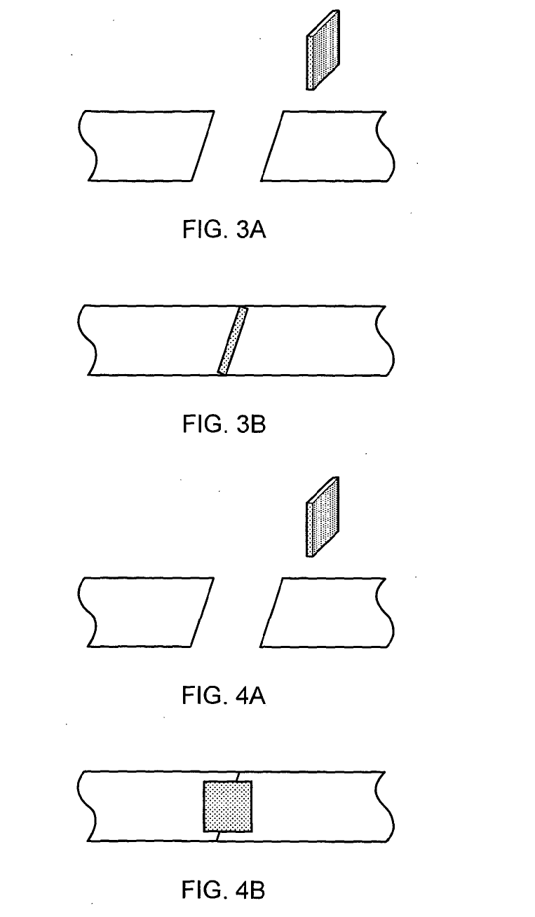

FIGS. 4A&B are schematic side views demonstrating an alternative use of the embodiment of FIGS. 3A&B;

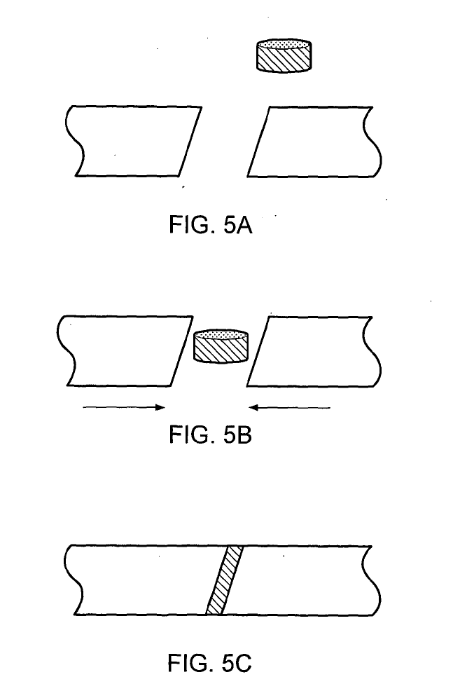

FIGS. 5A-C are schematic side views of an embodiment according to the first aspect of the invention; and

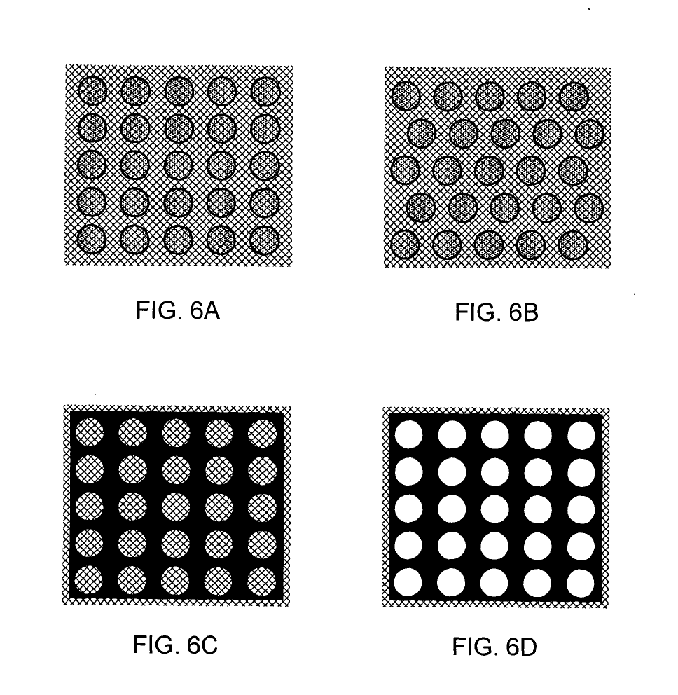

FIGS. 6A-D schematically represent different embodiments of the second aspect of the invention.

Referring to FIGS. 1 and 2, there is shown a schematic representation of a prior art capsule in the form of a syringe 10. Syringe 10 is formed of a tubular body 11, having an axial lumen, and a plunger 12. Plunger 12 fits tightly within the lumen and is slidably movable therein. Tubular body 11 is tapered at its distal end to form a spout 13, the distal end of which is sealed with a cap 14.

Tubular body 11 also includes a breakable seal 15 which divides the lumen into a first chamber 16, between a distal face of breakable seal 15 and spout 13, and a second chamber 21, between a proximal face of breakable seal 15 and the distal end of plunger 12.

The distal end of plunger 12 includes a seal 20 which provides the tight fit within lumen of tubular body 11. The plunger 12 can be pushed and pulled within the tubular body to expel or draw up a liquid or gas through spout 13, and also includes means for engaging and puncturing breakable seal 15 (not shown).

The first and second chambers 16 and 21 are respectively filled with an adhesive precursor and a liquid activator. In use, plunger 12 is used to break the breakable seal 15, causing the adhesive precursor and liquid activator to mix. Once seal 15 is broken, additional mixing of the two components is required, and this is carried out with suitable agitating means.

Subsequently, the cap 14 is removed and the mixture injected at the site of action, as shown in FIG. 2 where the mixed adhesive 17 is being applied to one surface of a pair of bone fragments 18.

Referring now to FIGS. 3A to 4B, schematic illustrations of embodiments according to the present invention are shown. For example, FIGS. 3A & B, show an adhesive precursor for fixing bone to bone which is activatable by endogenous aqueous fluids at the site of application, or by the addition of an aqueous solution. The adhesive precursor is provided as a coating on a substrate formed of fabric, paper or film, and may be coated on one or both faces of the substrate, either as a continuous coating or a discontinuous coating. The substrate and/or adhesive precursor may also be punctured with holes to allow tissue growth through the substrate (discussed in more detail below, in relation to FIGS. 6A-D).

The substrate is typically a fabric, which may be woven, knitted or non-woven—for example felt or an electrospun mat. The substrate may also be a tape. In preferred embodiments, the substrate is a resorbable polymer, such as polylactide, polyglycolide, poly(lactide-co-glycolide), polycaprolactone, polyurethane, a resorbable ceramic or glass, such as Bioglass or calcium phosphate, or any other suitable bioresorbable polymer, ceramic or glass. Alternatively, the substrate is a non-resorbable polymer, such as polyethylene terephthalate, polyethylene, polyurethane, polytetrafluroethylene, a non-resorbable ceramic or glass, such as Bioglass or calcium phosphate, or any other suitable bioresorbable polymer, ceramic or glass. The substrate may also be resorbable paper.

The coated/dipped substrate will ideally be provided in strips, or on a roll. When required, the substrate can be cut to a desired shape and size, and applied to one of the bone fragments before both, or all, of the bone fragments are brought together and held in a desirable configuration, as shown in FIG. 3B. Water contained in bodily fluids, and present at the site of application, will be absorbed by the adhesive precursor causing its activation to form a bone adhesive, in-situ. Alternatively, and to enhance fixation, particularly in repairs where very little endogenous fluids are present, the substrate may be dipped in an aqueous solution prior to its application. As a result, the bone fragments will be securely fixed together. As illustrated in FIGS. 3A&B, the substrate can be applied to the broken ends of the bone before these are brought together.

Alternatively, the substrate may be applied as a patch on the outer surface of the bone, as shown in FIGS. 4A&B. In further alternative embodiments, not shown, the substrate can be used like a tape and wound around a break in a bone.

In an alternative embodiment, shown in FIGS. 5A-C, the adhesive precursor is formulated as a tablet. The tablet may be formed with bulking agents and binding agents. In preferred embodiments, not shown, the tablet is formed as a thin sheet designed to minimise any impedance of the bone surfaces that are being joined together. In these preferred embodiments, the pressed sheet may also include a substrate in the form of a removable carrier formed of a film, foil or paper. The provision of the removable carrier allows the adhesive precursor to be more conveniently handled, and makes its application to bone fragments much more simple. The surgeon simply applies the adhesive precursor and carrier to the bone fragment, using the carrier to press the adhesive precursor against the bone, and waits for moisture at the site to activate the adhesive precursor. Once activation has started and the adhesive has begun to form, the carrier can be peeled away and the other bone fragments secured in place.

In further alternative embodiments, not shown, the adhesive precursor is applied directly as a powder.

To enhance the activation process of the adhesive precursor, or at application sites where very little endogenous fluid is present, additional water or a suitable aqueous solution can be used at the site of action. This can be introduced using any suitable means.

Referring to FIGS. 6A-D, there are shown a series of alternative arrangements of adhesive precursor and substrate. In particular, FIGS. 6A and 6B show a carrier in which adhesive precursor has been applied discontinuously in the form of “spots”. In an alternative arrangement shown in FIG. 6C, the adhesive precursor has been applied discontinuously with “holes” i.e. holes in adhesive precursor but not in substrate. In the further alternative arrangement of FIG. 6D, the adhesive precursor has been applied in a continuous manner and holes subsequently ‘punched’ through both the adhesive precursor and substrate. This arrangement is advantageous because it provides direct bone-bone contact which helps to promote the development of new bone tissue between the bone fragments.

The adhesive precursor is described above in the context of glass ionomer cements, but other suitable adhesive precursors such as calcium phosphates, zinc polycarboxylates, polyurethanes and other water-activated adhesive ceramic or polymer can be used.

In addition, the substrate and/or adhesive precursor may also include a porogen to further encourage tissue growth. Examples of porogens include water soluble salts, sugars, polymers etc

The adhesive precursor may also include a bioactive component to help stimulate tissue healing, particularly bone growth. Preferred examples of the bioactive components include antimicrobial compounds to help prevent, or to fight infection, growth factors such as bone morphogenetic proteins, enzymes, proteins or small molecules such as bisphosphonates.

The adhesive precursor is described in the context of bonding bone to bone, but could also be used to bond: metal implants to bone; bone cartilage/tendon/ligament/other soft tissue to bone or to itself; fill bone voids/defects using either a single piece of substrate comprising the adhesive precursor, or by building up layers; or to reinforce regions of bone to which implants are fixed, for example to reinforce screw holes in osteoporotic bone. The adhesive precursor could also be used in dentistry for craniomaxillofacial surgery.

### EXAMPLES

A glass-ionomer cement precursor powder was prepared by mixing glass XG153 (0.600 g) supplied by Advanced Healthcare Ltd, polyacrylic acid (0.200 g) and hydroxypropyl cellulose (0.016 g). (Glass:PAA ratio=3:1, with 2% hydroxypropyl cellulose wt %.)

1. Direct Application of Adhesive

A sample of GIC powder (0.80 g) blended as described above was mixed with 0.40 g of a 10% tartaric acid solution in water. The adhesive was applied directly to the cut ends of the bone and the two halves united and held together until the adhesive was set.

2. Application of Adhesive Precursor in the Form of Bandage or Tape (Between Bone Fragments)—FIGS. 3A&B

Preparation of Adhesive Precursor Coated Bandage/Tape/Mesh

The blended powders were then mixed with dichloromethane to form a slurry.

The slurry was then spread onto both sides of a piece of poly(glycolide-co-loctide) mesh ˜3 cm by 1 cm to form an even coating. The final coating weight was measured to be 960 g/m².

Sections of ovine tibia approximately 1 cm wide by 7 cm long were prepared and holes were drilled in the ends of each bone to allow subsequent mounting on a tensile testing machine. The bone samples were then cut into two approximately equal pieces roughly 1 cm wide by 3.5 cm long.

Pieces of the coated mesh described above were cut to approximate dimensions 1 cm by 0.5 cm. One of the cut bone surfaces was dampened with a drop of the 10% tartaric acid solution. The coated mesh was applied to the end of the bone and a further drop of tartaric acid solution applied to fully moisten it. The other end of the bone was then positioned and the two united pieces of bone held together until the adhesive had set.

3. Application of Adhesive Precursor in the Form of a Bandage or Tape (Wrapped Around Bone Fragments)—FIGS. 4A & B

Pieces of the coated mesh described above were cut to approximate dimensions 1 cm by 1 cm. The two ends of a cut bone were brought into contact and the outer surface of the bone around the cut was dampened with the 10% tartaric acid solution. The coated mesh was applied to the outer surface of the bone and dampened with another drop of tartaric acid solution. The coated mesh was then moulded to the contours of the bone and the pieces held together until set.

In all cases the adhesive set within 5 minutes. The bonded bone samples were wrapped in damp paper towels and stored in sealed polythene bags at 37 C for approximately 24 hours prior to testing.

After 24 hours the bond strengths were measured in tension using an Instron tensile testing machine. Steel pins were placed through the holes in the bone ends to allow mounting in a testing rig. The samples were tested in tension at a cross-head speed of 5 mm/min.

4. Application of Adhesive Precursor in Powder Form

i) A control glass-ionomer cement adhesive was prepared by stirring glass 1A1SrZn20* (0.200 g), polyacrylic acid (0.075 g) and water (0.075 g) to form a paste.
*Composition of Glass 1A1SrZn20

Glass 1A1SrZn20 was made at Imperial College, London, by combining the materials shown in the table below in the weight percentage shown.

| Glass | SiO₂ | CaO | CaF₂ | SrO | SrF₂ | MgO | ZnO |
|---|---|---|---|---|---|---|---|
| 1A1SrZn20 | 47.7 | 5.3 | 5.6 | 5.3 | 4.6 | 12.4 | 19.1 |

The adhesive was used to join two pieces of ovine tibia as described above, applying the adhesive to the cut ends of the bone. The cross-sectional areas of the cut ends of the bone were approximately 40-45 mm². The adhesive set in ˜20 minutes.

ii) A GIC precursor powder was prepared by mixing glass 1A1SrZn20 (0.200 g) with polyacrylic acid (0.075 g).

Samples of ovine tibias were prepared as above. The cross-sectional areas of the cut ends of the bone were approximately 40-45 mm². The cut ends of the bone were wetted with water and the GIC powder applied. The ends of the bone were pushed together to form a bond and a few additional drops of water were applied. The provider absorbed the water and the adhesive set in around 10 minutes.

Subsequently, the bone samples were wrapped in damp paper towels and stored at 37 C for 2 hours, after which time they were removed and the bonds tested in tension using a spring balance to measure the failure load.

5. Application of Adhesive Precursor in the Form of a Tablet (Between Bone Fragments)—FIGS. 5A-C

Preparation of Adhesive Precursor Tablets

0.24 g zinc oxide was mixed with 0.24 g polyacrylic acid and 0.01 g hydroxypropyl cellulose. The blended powders were mixed with dichloromethane (approximately 50:50) to form a slurry. The slurry was spread over a PTFE sheet (approximately 0.50 mm thick) which contained an array of 4 mm diameter holes punched in it. The solvent was allowed to evaporate and then the discs of adhesive precursor, thus formed, were pushed out of the holes to form small tablets.

The tablets were used to bond pieces of ovine cortical bone from the tibia as described previously. Two tablets of adhesive precursor were placed onto the end of one of the bone fragments. These were then wetted with a small drop of water to activate the adhesive, and the two pieces of bone were brought together and held for approximately 1 minute until firmly stuck. The bonded bone samples were placed in phosphate buffered saline solution at 37° C. for approximately 24 hours and then subjected to a tensile test as described previously.

Results

| | Mean Failure | Mean Failure |
|---|---|---|
| Sample | Load (N) | Stress (MPa) |
| 1. Adhesive only (control) | 86.1 | 1.94 |
| 2. Coated mesh applied | 78.7 | 2.08 |
| to bone ends | | |
| 3. Coated mesh applied | 42.7 | N/A |
| to side of bone | | |

These results show that when applied to the bone ends the adhesive applied on the mesh is just as effective as the adhesive applied in the conventional way. Furthermore, a good bond strength can also be achieved by applying the coated mesh to the side of the bone.

The adhesive pre-mixed with water in the conventional way [4(i)] gave an average failure load of 1.9 kg. The adhesive precursor applied as a powder and activated with water in-situ [4(ii)] gave an average failure load of 1.4 kg. The adhesive precursor applied as a tablet [5] gave an average tensile bond strength of 1.2 MPa.

The described invention advantageously permits the use of very reactive, fast-setting, adhesive systems. It is particularly advantageous as it avoids the need for the surgeon to mix separate components, prior to application, and allows the use of compositions with very short working/setting times which may otherwise be impractical to use. Furthermore, the amount and distribution of the adhesive within the site of application can readily be controlled by cutting the tape or bandage coated with adhesive precursor to the required size, or using an appropriately sized tablet. By using a configuration of the adhesive on the carrier with holes or gaps bone healing can be promoted. The system of the invention is particularly useful as it can be applied to both the surfaces to be joined, or the sides of the bone:

What is claimed is:

1. An adhesive precursor for securing bone to bone, wherein the adhesive precursor is directly applicable to bone and is activated in-situ by endogenous aqueous fluids.

2. An adhesive precursor according to claim 1, wherein adhesive precursor is a powder.

3. An adhesive precursor according to claim 2, wherein the powder is formed, with or without bulking agents, as a tablet or a pressed sheet.

4. An adhesive precursor according to claim 1, wherein the adhesive precursor is a viscous liquid.

5. An adhesive precursor according to claim 1, wherein the adhesive precursor is a glass ionomer cement, calcium phosphate, zinc polycarboxylate, polyurethane or other water-activated adhesive ceramic or polymer.

6. An adhesive precursor according to claim 5, wherein the adhesive precursor is a glass ionomer cement.

7. An adhesive precursor according to claim 1, further comprising a substrate.

8. An adhesive precursor according to claim 7, wherein the substrate is a tape, film, paper, foil or fabric.

9. An adhesive precursor according to claim 8, wherein the substrate is a removable paper, foil or film.

10. An adhesive precursor according to claim 8, wherein the substrate is a fabric.

11. An adhesive precursor according to claim 10, wherein the fabric is knitted, woven, spun or non-woven.

12. An adhesive precursor according to claim 7, wherein the substrate is bioabsorbable.

13. An adhesive precursor according to claim 7, wherein the substrate is non-resorbable.

14. An adhesive precursor according to claim 12, wherein the bioabsorbable substrate is a polylactide, polyglycolide, poly(lactide-co-glycolide), polycaprolactone, polyurethane, or other bioresorbable polymer, or a ceramic or glass such as Bioglass, calcium phosphate, or another bioabsorbable ceramic/glass.

15. An adhesive precursor according to claim 13, wherein the non-resorbable substrate is a polyethylene terephthalate, polyethylene, polyurethane, polytetrafluroethylene, or other non-resorbable biocompatible polymer, and ceramic or glass such as Bioglass, calcium phosphate, or other non-bioresorbable ceramic/glass.

16. An adhesive precursor according to claim 7, wherein the adhesive precursor is applied to one side or both sides of the substrate.

17. An adhesive precursor according to claim 16, wherein the adhesive precursor is applied as a continuous coating or as a non-continuous coating.

18. An adhesive precursor according to claim 1, wherein the substrate and/or adhesive precursor includes a plurality of holes.

19. An adhesive precursor according to claim 1, further comprising a porogen.

20. An adhesive precursor according to claim 19, wherein the porogen is a water-soluble salt, a sugar or a polymer.

21. An adhesive precursor according to claim 1, further comprising a bioactive component.

22. An adhesive precursor according to claim 21, wherein the bioactive component is an antimicrobial compound, growth factor, enzyme, protein or small molecule.

23. An adhesive precursor according to claim 22, wherein the bioactive component is a bone morphogenetic protein.

24. An adhesive precursor according to claim 22, wherein the bioactive component is a bisphosphonate.

25. A method of joining bone fragments wherein the method comprises the steps of:
a) providing an adhesive precursor according to any one of the preceding claims; and
b) (i) applying the adhesive precursor directly to one or more of the bone fragments to be combined, and
(ii) bringing the bone fragments together; or c) (i) bringing the bone fragments together, and
(ii) applying the adhesive precursor to one or more outer surfaces of the bone fragments to be combined;
wherein the adhesive precursor is activated in-situ by endogenous aqueous fluids which are present at the site of application.

26. A method according to claim 25, further comprising the step of applying an aqueous solution to the adhesive precursor prior to its application to the bone fragments
